(19) United States
(12) Patent Application Publication
(10) Pub. No.: US 2009/0238900 A1
(43) Pub. Date: Sep. 24, 2009

(54) NOVEL ANTI-DIABETIC HERBAL COMPOSITION, METHOD FOR PREPARING THE SAME AND USE THEREOF

(75) Inventors: Shankar Kumar Mitra, Bangalore (IN); Ekta Saxena, Bangalore (IN); Uddagiri Venkanna Babu, Bangalore (IN)

Correspondence Address: KNOBBE MARTENS OLSON & BEAR LLP, 2040 MAIN STREET, FOURTEENTH FLOOR, IRVINE, CA, 92614, US

(73) Assignee: HIMALAYA GLOBAL HOLDINGS LIMITED, Grand Cayman, KY (US)

(21) Appl. No.: 12/199,621

(22) Filed: Aug. 27, 2008

(30) Foreign Application Priority Data
Mar. 24, 2008 (IN) 734/DEL/2008

(51) Int. Cl. A61K 36/00; A61P 3/10

(52) U.S. Cl. 424/725

(57) ABSTRACT

Disclosed is an herbal composition for the treatment of Type II diabetes and their associated secondary complications in humans, wherein said composition comprising extract of roots of Cipadessa baccifera and pharmaceutically acceptable excipients, method for preparing the same and use thereof. The anti-diabetic herbal composition disclosed herein is effective to reduce the glucose, triglyceride levels in blood or other complications related to Type-II diabetes and its secondary complications thereof.

### FIELD OF THE INVENTION

This invention relates to a novel anti-diabetic herbal composition comprising extract of roots of Cipadessa baccifera and pharmaceutically acceptable carrier, methods for preparing the same and use thereof.

### BACKGROUND OF THE INVENTION

Diabetes mellitus is a metabolic disorder of multiple etiologies characterized by chronic hyperglycemia with disturbed carbohydrate, fat and protein metabolism resulting from defects in either insulin secretion or insulin action or both. In Type I diabetes, the mammal's ability to store glucose is reduced due to reduced insulin production whereas, Type II diabetes or Non-Insulin Dependent Diabetes Mellitus (NIDDM) is caused by resistance to insulin stimulating or regulating effect on glucose and lipid metabolism in the main insulin-sensitive tissues, liver, muscle and adipose tissue. Around 90-95% of diabetic patients are belonging to Type-II condition among the total diabetic patients existing worldwide.

The resistance to insulin responsiveness causes insufficient insulin activation of glucose uptake, oxidation and storage in muscle and inadequate insulin repression of lipolysis in adipose tissues and of glucose production and secretion in liver.

Patients with Type-II diabetes suffer from both reduced insulin secretion and from resistance to the actions of insulin. In normal case, insulin controls the blood glucose concentration by stimulation of glucose uptake in specific insulin sensitive tissues. Skeletal muscle glucose uptake is an important determinant of glucose homeostasis, since muscle is the largest tissue in the body and a major site of insulin regulated glucose clearance. The glucose transporters, GLUT1 and GLUT4 coexist in tissues, wherein, the glucose transport is markedly stimulated by insulin. Several lines of evidence suggest that GLUT 4 is the major glucose transporter in muscle cells. Insulin resistance in Type-II diabetes is manifested by decreased insulin stimulated glucose transport and metabolism in adipocytes and skeletal muscle resulting in down regulation of the major insulin responsive glucose transporter GLUT4. Peroxisome Proliferator Activated Receptor (PPARγ) agonist thiazolidinediones are insulin-sensitizing agents and are used in the treatment of Type-II diabetes. It is known that PPARγ regulates glucose metabolism by increasing glucose uptake through facilitative GLUTs.

Although there are many medications available to treat diabetes, all these treatments remain unsatisfactory due to side affects and their limitations. As it is difficult to maintain adequate glycemic control in patients over a time with Type-II diabetes, the interest in insulin sensitizers is growing in Type-II diabetic patients. Recently, it is evident that the PPARγ agonist, the insulin sensitizer, may have benefits in the treatment of Type-II diabetes, apart from their effects in improving glycemic control.

There are many herbal medications reported to control diabetes but only few of them are studied in detail. It is therefore necessary to evaluate a large number of herbal drugs available for diabetes and their mechanism of action at cellular and molecular level for our better understanding this in turn would lead to better treatment options for Type-II diabetes and their secondary complications.

### RELATED ART

United States Patent Application No. 20070276138 discloses a method of preparation of modulators of Peroxisome Proliferators Activated Receptors (PPAR) and their importance in the treatment of Type-II diabetes.

United States Patent Application No. 20070249519 teaches a method of preparation of a composition for upregulation of GLUT4 via modulation of PPAR δ in adipose tissue and for the treatment of disease.

Anandharajan et. al. revealed about the in vitro glucose uptake activity of Aegles marmelos and Syzygium cumini by activation of GLUT4, P13 kinase and PPARγ in L6 myotubes (Phytomedicine, 13, 2006, 434-441)

Gan L S et. al. unveiled about the isolation and characterization of Tetranortriterpenoids from the seeds of Cipadessa baccifera (J. Nat. Prod., 70(8), 2007, 1344-47).

Luo X D et. al. discloses about the isolation and characterization of four compounds from the seeds of Cipadessa baccifera (Phytochemistry, 55(8), 2000, 867-72).

### SUMMARY OF THE INVENTION

It is a principal aspect of the present invention to provide a novel herbal composition for treatment of Type-II diabetes and secondary complications thereof comprising extracts of roots of Cipadessa baccifera and pharmaceutically acceptable excipients.

In accordance with another aspect of the present invention, there is provided a novel anti-diabetic herbal composition for treatment of Type-II diabetes and secondary complications thereof, wherein said composition is effective to decrease the glucose and triglyceride levels in blood.

In accordance with one another aspect of the present invention, there is provided a novel anti-diabetic herbal composition for treatment of Type-II diabetes and secondary complications thereof, wherein the extract can be obtained from all or any specific part/s of Cipadessa baccifera, preferably employing roots of Cipadessa baccifera.

In accordance with yet another aspect of the present invention, there is provided a method of extraction from roots of Cipadessa baccifera, wherein said extraction method is performed using any suitable extraction technique preferably a hot soxhlation or percolation technique and wherein a selective solvent or solvent system are used to yield high extractive value and rich content of stable bioactive ingredients. Said solvent is selected from a group of organic solvents and/or water, preferably water or butanol alone or in combination.

In accordance with yet another aspect of the present invention, there is provided a novel anti-diabetic herbal composition for treatment of Type-II diabetes and secondary complications thereof, wherein resultant extract of roots of Cipadessa baccifera contains an active fraction capable of reducing the glucose and triglyceride levels in blood or other complications related to the Type-II diabetes.

In accordance with a further aspect of the present invention, there is provided an herbal diabetic composition, wherein said composition is formulated as various delivery systems including but are not limited to tablet, capsules, syrup, cream or gel.

In yet another aspect of the present invention, there is provided a therapeutic amount of active extract of roots of Cipadessa baccifera is about 100 mg to about 1000 mg.

### BRIEF DESCRIPTION OF THE DRAWINGS

Further objects of the present invention together with additional features contributing thereto and advantages accruing there from will be apparent from the description of preferred embodiments of the present invention which are shown in the accompanying drawing figures.

FIG. 1 shows effect of GP-extracts on glucose uptake in differentiated L6 cells.

FIG. 2 shows effect of GP-10 on glucose uptake in differentiated L6 cells in different concentrations.

FIG. 3 shows effect of GP-10 fractions on glucose uptake in differentiated L6 cells in different concentrations.

FIG. 4 shows effect of GP-10/1 chromatographic fractions on glucose uptake in differentiated L6 cells at 200 μg/ml concentration.

FIG. 5 shows effect of E series test drugs on glucose uptake in differentiated L6 cells.

FIG. 6 shows densitometric analysis of a representative data of PPARγ transcripts respectively in comparison with GAPDH gene.

FIG. 7 shows effect of GP-10 at various concentration on PPARγ gene expression in L6 cells. Analysis of GAPDH transcripts in L6 was also carried out.

FIG. 8 shows densitometric analysis of a representative data of GLUT4 transcripts respectively in comparison with GAPDH gene.

FIG. 9 shows effect of GP-10 at various concentrations on GLUT-4 gene expression in L6 cells. Analysis of GAPDH transcripts in L6 was also carried out.

FIG. 10 shows effect of GP-10 on Oral glucose tolerance test in normal rats (OGTT) (Day-1).

FIG. 11 shows effect of GP-10 on Oral glucose tolerance test in normal rats (OGTT) (Day-8).

FIG. 12 shows hypoglycemic activity of GP-10 in STZ diabetic rats.

FIG. 13 shows hypoglycemic activity of GP-10 in STZ diabetic rats (after 8 days of treatment).

FIG. 14 shows effect of GP-10 on Fructose induced insulin Resistance in Rats (Day 21).

FIG. 15 shows effect of GP-10 on Fructose induced Insulin Resistance in Rats (Day 21).

FIG. 16 shows effect of GP-10 on Fructose induced Insulin Resistance in Rats (Day 42).

FIG. 17 shows effect of GP-10 on Fructose induced Insulin Resistance in Rats (Day 42).

FIG. 18 shows effect of GP-10 on Blood TG level of Fructose fed rats (4 hr fasted animals).

FIG. 19 shows effect of GP-10 on Blood Glucose Level of Fructose fed rats (4 hr fasted animals).

FIG. 20 shows effect of GP-10 on oral glucose tolerance in fructose fed rats.

FIG. 21 shows HPLC Chromatogram of water extract of Cipadessa baccifera.

FIG. 22 shows HPLC Chromatogram of methanol fraction of water extract of Cipadessa baccifera.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The Cipadessa baccifera Roth disclosed herein belongs to family Meliaceae is a shrub or small tree and distributed in the Indo-Malaysian region and wildly found in India. It is a highly branched shrub, up to 2.5 m in height with imparipinnate leaves, elliptic lanceolate leaflets, small white flowers in axillary corymbose panicles, and red globose berries and is commonly found in parts of South India, Bihar, and Orissa and in the eastern Himalayas up to a height of 1,500 m. The roots of the plant are used against tapeworms, the leaves as a poor fodder and the wood as a fuel.

The present invention provides an anti-diabetic herbal composition effective for treatment of Type II diabetes and associated secondary complications thereof. In addition, the disclosed anti-diabetic herbal composition is effective in reducing the blood level of glucose and triglyceride in fructose fed rats. Further, the disclosed anti-diabetic herbal composition exhibits change in glucose tolerance level in fructose fed rats and blood glucose levels of streptozotocin (STZ) induced rats. The disclosed herbal diabetic composition, according to the present invention contains dried water extract of roots of Cipadessa baccifera, wherein said extract is dried by any suitable drying techniques such as freeze drying or spray drying to obtain free flowing powder of water extract of roots of Cipadessa baccifera.

The biologically active fraction of water extract of roots of Cipadessa baccifera is purified by column chromatographic technique. Moreover, the obtained purified fraction of the extract is standardized by High Performance Column Chromatography (HPLC).

In accordance with one exemplary embodiment of the present invention, the water extract of roots of Cipadessa baccifera is subjected to solvent-solvent fractionation employing methanol, butanol and/or water alone or in-combination thereof. These fractions are subjected to in vitro glucose uptake activity, particularly, evaluated for glucose transport in L6 muscle cell. Further, the water extract of roots of Cipadessa baccifera and its fractions are evaluated for glucose uptake activity.

According to the present invention, the water extract of roots of Cipadessa baccifera is evaluated for oral glucose tolerance in fructose-induced insulin resistant rats. Further, the hypoglycemic activity of water extract of roots of Cipadessa baccifera is evaluated by using Streptozotocin induced human model.

The disclosed herbal diabetic composition according to the present invention comprises the pharmaceutically acceptable excipients selected from, but are not limited to filler, binder, disintegrating agent, lubricant, preservative, sequestering agent, thickening agent, solvent, flavoring agent, sweetening agent, humectant, rheology modifier, emollient, emulsifying agent and/or water. Preferably, selected from but are not limited to lactose, microcrystalline cellulose, pre-gelatinized starch, magnesium stearate, sorbitol, sodium citrate, citric acid, stearic acid, disodium EDTA, xanthan gum, propylene glycol, mint flavor, sodium saccharin, alkyl parabens, glycerin, corbopols, cetyl alcohol and/or water. The disclosed herbal anti-diabetic composition, according to the present invention is formulated as various delivery systems including but are not limited to tablet, capsules, syrup, cream or gel.

The following non-limiting examples illustrate specific embodiments of the present invention. They are, not intended to be limiting the scope of present invention in any way.

### EXAMPLE-1

### Preparation of Extract from Cipadessa Baccifera by Percolation Method

The shade dried roots of Cipadessa baccifera were pulverized to coarse powder, about 5 Kg of powdered material placed in different flasks and extracted with n-hexane, dichloromethane, chloroform, ethyl acetate, acetone, ethanol, methanol, water, methanol and water (1:1) and acetone and water (1:1) at room temperature for 24 h to 48 h, then plant extracts were filtered and concentrated to dryness on rotatory evaporator or on steam bath at optimum temperature and under reduced pressure. The yields of extracts so obtained are mentioned in Table-1.

### EXAMPLE-2

### Preparation of Extract from Cipadessa Baccifera by Hot-Soxlation Method

The coarse powdered material of roots of Cipadessa baccifera was subjected to hot-soxlation using solvents n-hexane, dichloromethane, chloroform, ethyl acetate, acetone, ethanol, methanol, water, methanol and water (1:1) and acetone and water (1:1) at optimum temperature and recycled until extraction is completed, then plant extracts were filtered and concentrated to dryness on rotatory evaporator or on a steam bath at optimum temperature. All extracts were qualitatively similar to extracts prepared by percolation method. The yields of extracts so obtained are mentioned in Table-1.

TABLE 1
Yields of extracts of Cipadessa baccifera

| | | Nature of Solvent | Yield (%) | |
|---|---|---|---|---|
| S. No | Code No | Extraction | Percolation | Soxhalation |
| 1 | GP-01 | Hexane | 3.8 | 4.2 |
| 2 | GP-02 | Chloroform | 5.7 | 6.2 |
| 3 | GP-03 | Ethylacetate | 4.7 | 4.9 |
| 4 | GP-04 | Dichloromethane | 4.3 | 5.0 |
| 5 | GP-05 | Methanol | 10.0 | 12.0 |
| 6 | GP-06 | Ethyl Alcohol | 7.0 | 7.5 |
| 7 | GP-07 | Acetone | 6.2 | 6.5 |
| 8 | GP-08 | Methanol:water | 6.9 | 7.0 |
| 9 | GP-09 | Acetone:water | 9.0 | 9.5 |
| 10 | GP-10* | Water | 6.6 | 75 |

*GP-10 is synonymously used as E2 in the specification.

### EXAMPLE-3

### Solvent Fractionation of Water Extract (GP-10)

The water extract of roots of Cipadessa baccifera was subjected to solvent-solvent fractionation with methanol, methanol-water (90:10), methanol-water (25:75), methanol-water (75:25), methanol-water (50:50) and water-soluble fractions. All these fractions were submitted for in vitro glucose uptake activity. The yields of fractions are given in Table-2.

TABLE 2
Yields of fractions of Water extract (GP-10)

| S. No | Solvent used | Code no | Yield (%) |
|---|---|---|---|
| 1 | Methanol | GP-10/1 | 55% |
| 2 | Methanol:Water (90:10) | GP-10/2 | 10% |
| 3 | Methanol:Water (25:75) | GP-10/3 | 02% |
| 4 | Methanol:Water (75:25) | GP-10/4 | 10% |
| 5 | Methanol:Water (50:50) | GP-10/5 | 10% |
| 6 | Water | GP-10/6 | 05% |

### EXAMPLE-4

### n-Butanol/Water Partition of Water Extract GP-10

About 500 g of water extract (E2) was dissolved in 3.5 L of double distilled water and filtered through 100# muslin cloth. The filterate was partitioned between water and n-Butanol (1 L) and the n-Butanol layer was separated and the partition was performed three times and all the n-Butanol layers were mixed and concentrated to a dry mass on rotatory evaporator under reduced pressure to give n-butanol fraction (E3) and water fraction (E4).

### EXAMPLE-5

### Spray Drying of Water Extract GP-10

Approximately 5 Kg of water extract (GP-10) prepared by percolation method and containing 30% solids was subjected to spray drying at a flow rate of 1 L/hour with the maximum in-let temperature of 140-160° C. and out-let temperature 103° C. to give free flowing dry extract powder. The HPTLC of dry extract powder was carried out with water extract of percolation method and found to be similar to water extract prepared by percolation method.

### EXAMPLE-6

### Freeze-Drying of Water Extract GP-10

Approximately 5 Kg of water extract (GP-10) was subjected to freeze drying in a tray of 1.1 meter length and 0.57 meters width under vacuum at 0.6 Torr and temperature maintained at −37° C. to give free flowing dry extract powder. The HPTLC of freeze dried extract powder was carried out with water extract of percolation method and found to be similar to water extract prepared by percolation method.

### EXAMPLE-7

### Column chromatography of GP-10/1 Fraction of GP-10 Extract

Approximately 20 g of methanol soluble fraction of water extract (GP-10) of roots of Cipadessa baccifera was subjected to column chromatography over silica gel (60-120 mesh) and eluted with n-hexane and ethyl acetate and ethyl acetate and methanol with increasing polarity and fractions of 100 ml each were collected and pooled accordingly based on thin layer chromatography analysis. All similar fractions were combined and concentrated to give subfractions. The details of these fractions and their yields are given in table-3. All these fractions were subjected to in vitro Glucose uptake activity.

TABLE 3
Details of column chromatography of GP-10/1 fraction

| | Fraction | | | Yield |
|---|---|---|---|---|
| S. No. | No | Code No | Sovent eluant | (mg) |
| 1 | 01-40 | ESG-75 | Hexane:ethylacetate 10% | 500 |
| 2 | 41-48 | ESG-76 | Hexane:ethylacetate 20% | 500 |
| 3 | 49-57 | ESG-77 | Hexane:ethylacetate 20% | 150 |
| 4 | 58-66 | ESG-78 | Hexane:ethylacetate 40% | 500 |
| 5 | 67-76 | ESG-79 | Hexane:ethylacetate 60% | 307 |
| 6 | 77-84 | ESG-80 | Hexane:ethylacetate 80% | 220 |
| 7 | 85-88 | ESG-81 | Ethylaceate (100%) | 500 |
| 8 | 89-92 | ESG-82 | Ethylacetate:methanol (10%) | 520 |
| 9 | 93-96 | ESG-83 | Ethylacetate:methanol (10%) | 1000 |
| 10 | 97-100 | ESG-84 | Ethylacetate:methanol (20%) | 2000 |
| 11 | 101-107 | ESG-85 | Ethylacetate:methanol (40%) | 4000 |
| 12 | 108-113 | ESG-86 | Ethylacetate:methanol (60%) | 2000 |
| 13 | 114-120 | ESG-87 | Ethylacetate:methanol (80%) | 3000 |
| 14 | 121-126 | ESG-88 | Methanol (100%) | 500 |

### EXAMPLE-8

### In vitro Analysis of Herbal Extract GP-10 and Its Fractions on Glucose Transport in L6 Muscle Cells

### Materials and Methods

Chemicals and reagents: Dulbecco's Modified Eagle Medium (DMEM), Fetal Bovine Serum (FBS), Bovine Serum Albumin (BSA), 3-(4,5-dimethylthiazol-2-yl)-2,5 diphenyltetrazolium bromide (MTT), Cytochalasin B and 2-deoxy glucose were procured from Sigma Aldrich, St. Louis. ¹⁴C 2-deoxy glucose was obtained from BRIT, Mumbai. Insulin (Torrent Pharmaceuticals, 40 IU/ml) was purchased from a drug store. Rosiglitazone (RTZ) was procured from Cayman Chemical Company, Ann Arbor. Penicillin and Streptomycin solution was purchased from Hi Media Labs, Mumbai. All the other chemicals used were of the highest analytical grade. Culture flasks and plates were purchased from Tarsons Pvt Ltd, Kolkata.

Herbal Extracts and drug dilutions: Extracts and fractions were weighed and dissolved initially in suitable solvent and stock solution of 5 mg/ml was prepared using DMEM containing 2% FBS/KKRP buffer. This was further diluted with same medium to attain further lower dilutions. All the dilutions were prepared fresh each time. Dilutions prepared in DMEM with 2% FBS were used for cytotoxicity studies and dilutions with KRP buffer were used in glucose uptake assay.

Cell culture: The L6 skeletal muscle cells were procured from National Center for Cell Sciences, Pune, India. Cells were cultured and maintained in DMEM with 10% FBS and supplemented with Penicillin (100 IU/ml) and Streptomycin (100 μg/ml) at 37° C. with 5% CO₂ atmosphere. For differentiation, L6 cells were maintained in DDMFM with 2% FBS for 4-6 days post confluence. The extent of differentiation was established by observing multinucleation of cells.

Cytotoxicity studies: The 24 hr cell cultures with 70-80% confluency in 96 well plates were used for the study. 100 μL of each dilution of the test drugs were added in quadruplicate in 96 well plate and cell controls maintained in same number. The cultures were incubated at 37° C. with 5% CO₂ for 24 hrs and the cultures were observed microscopically for any visible change in morphology of cells and observations were recorded. The cell viability assay was determined by MTT assay. The percentage cytotoxicity caused by each dilution of the drug was determined and Cytotoxic Concentration 50 (CTC₅₀) values determined by interpolation method. The non-toxic concentrations of test drugs, i.e. concentrations below CTC₅₀ value were taken for glucose uptake studies.

Glucose uptake assay: Glucose uptake activity of test drugs was determined in differentiated L6 cells. In brief the differentiated cells were serum starved over night and at the time of experiment cells were washed with HEPES buffered Krebs Ringer Phosphate solution (KRP buffer) once and incubated with KRP buffer with 0.1% BSA for 30 min at 37° C. Cells were treated with different non-toxic concentrations (about 200 μg/ml) of test and standard drugs for 30 min along with negative controls at 37° C. 20 μl of 0.1 to 5 μCi of ¹⁴C 2-deoxy-glucose solution and 0.1 to 1 mM of 2-deoxy glucose solution were added simultaneously to each well and incubated at 37° C. for 30 min. After incubation, the uptake of the glucose was terminated by aspiration of solutions from wells and washing thrice with ice-cold KRP buffer solution. Cells were lysed with 0.1M NaOH solution and an aliquot of cell lysates were used to measure the cell-associated radioactivity by liquid scintillation counting using Packard Top count NXT. Three to four independent experimental values in duplicates were taken to determine the percentage enhancement of glucose uptake over controls. Data is analyzed by non-parametric Student t-test using GraphPad Prism4.

Reverse transcriptase polymerase chain reaction: To investigate the mechanism of action of GP-10 in glucose uptake, treated cells were analyzed for PPARγ and GLUT4 gene expression by Reverse transcriptase PCR (RT-PCR) as described previously. In brief, L6 cells were treated with different concentrations of GP-10 along with standard drugs rosiglitazone and insulin in DMEM with 2% FBS at 37° C. with 5% CO₂ for 24 h. After incubation, media was aspirated and total RNA was extracted from cells using TRI reagent (Sigma). The isolated RNA was quantified on denaturing agarose gel and was subjected to Dnase I treatment. Total RNA and oligo dT were used for cDNA synthesis by Reverse transcriptase (Fermetas, USA). PCR amplification was carried out using specific primers (Sigma, USA). The primers used were as follows: Glut-4 sense 5′-CGG GAC GTG GAG CTG GCC GAG GAG-3′; antisense-5′-CCC CCT CAG CAG CGA GTG A-3′ PPARγ sense 5′-GGA TTC ATG ACC AGG GAG TTC CTC-3′; anti-sense, 5′-GCG GTC TCC ACT GAG AAT AAT GAC-3′ and GAPDH sense, 5′-CCA CCC ATG GCA AAT TCC ATG GCA-3′; anti-sense, 5′-TCT AGA CGG CAG GTC AGC TCC ACC-3′. For PPARγ gene amplification the PCR reaction was carried out for 35 cycles, with each cycle comprising a denaturation for 20 sec at 95° C., annealing for 30 sec at 60° C. and elongation for 40 sec at 72° C. For GLUT4 gene amplification, the PCR reaction was carried out for 30 cycles with each cycle comprising a denaturation for 45 sec at 95° C., annealing for 1 min at 65° C. and elongation for 1 min at 72° C. The final extension was carried out at 72° C. for 10 min. GAPDH was used as internal control in each case. The final PCR products were resolved on 1.5% agarose gel stained with ethidium bromide and photographed under exposure to UV light. The molecular weight of the amplified cDNA was determined by comparison with a standard molecular weight marker (1 Kb ladder) and densitometry analysis was carried out. Data is representative of one experiment.

### Results

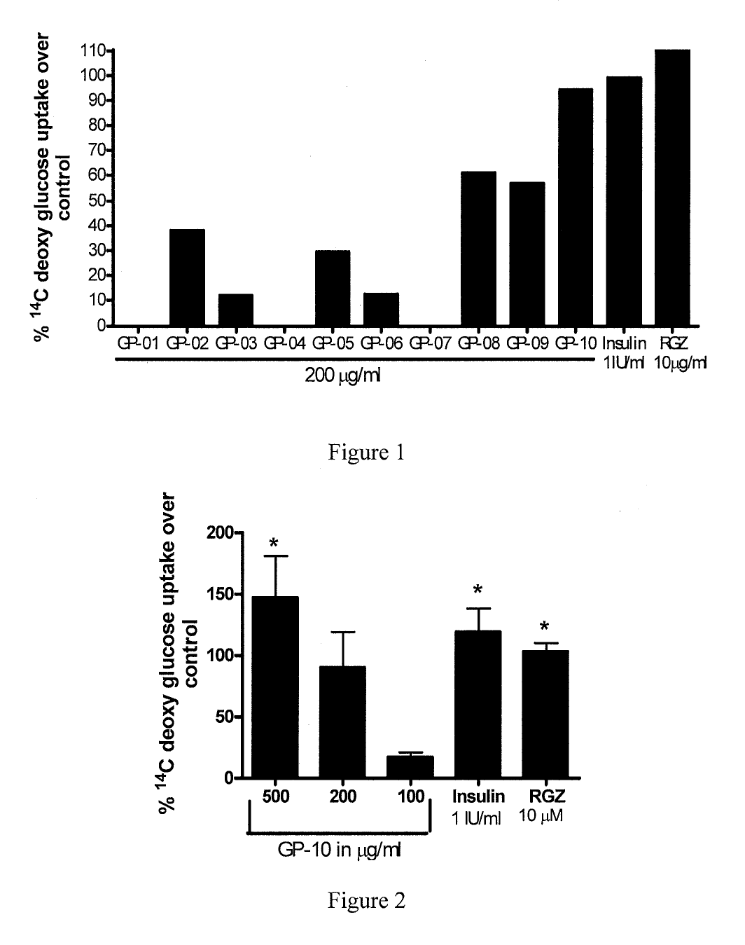

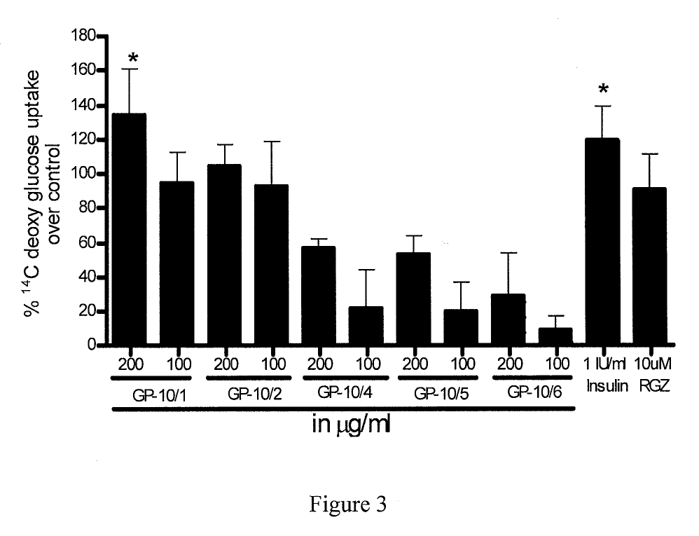

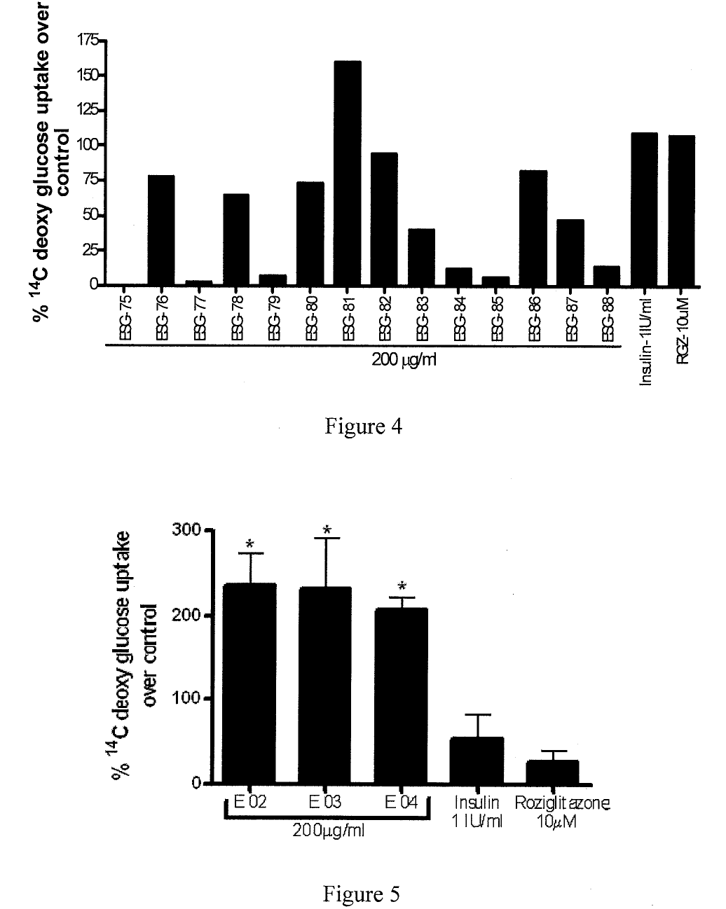

Glucose uptake enhancement efficacy of GP-10 and its fractions: A preliminary in vitro study of crude extracts in L6 cells showed that the enhanced glucose uptake activity was found in GP-10 among all other extracts GP-01 to GP-09 (FIG. 1). The GP-10 extract exhibited 146.70 and 94.07 percent glucose uptake enhancement at varying concentration of 500 and 200 μg/ml respectively (FIG. 2) and was taken for further fractionation. The fractions obtained from GP-10 were further subjected to glucose uptake assay at varying concentrations and the fraction GP-10/1 exhibited significant percentage enhancement in glucose uptake at 200 μg/ml whereas other fractions exhibited moderate glucose uptake activity (FIG. 3). Based on these findings, GP-10/1 was taken for further purification by column chromatography and semi purified fractions ESG-75 to ESG-88 were evaluated for their glucose uptake enhancement efficacy at three nontoxic dose levels. From the results, fraction ESG-81 was found to have potent glucose uptake activity (FIG. 4) followed by ESG-82 and ESG-76. In another fractionation method, GP-10 extract (E2) was subjected to solvent-solvent fractionation between n-butanol and water and the fractions n-butanol (E3) and water (E4) were subjected to glucose uptake and results are shown in FIG. 5.

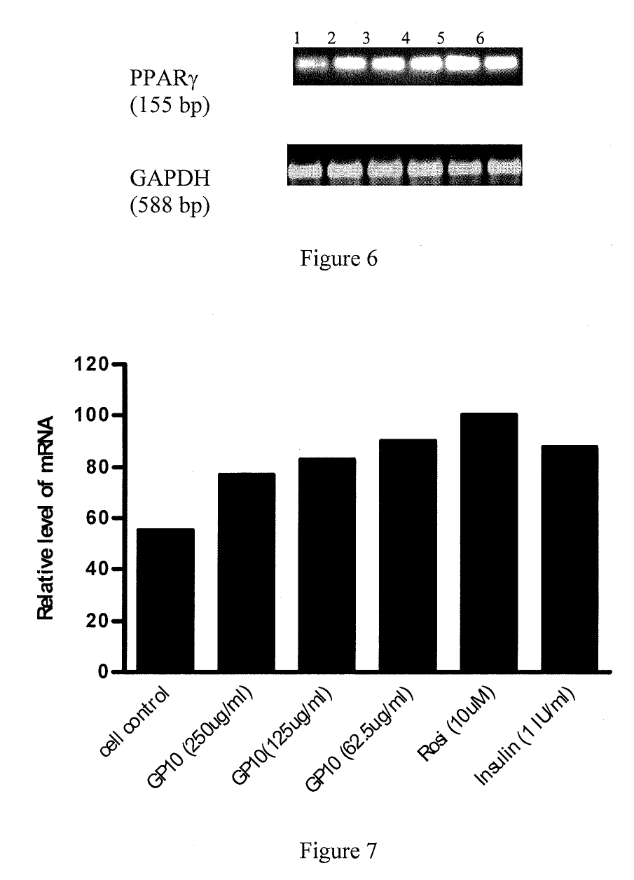

Modulation of GP-1 on PPARγ and GLUT4 gene expression by GP-10: Our observations from RT-PCR analysis indicated elevated expression of PPARγ transcripts by GP-10 at all the test concentrations on par with rosiglitazone and insulin (FIG. 6). GAPDH was used as internal control and was uniformly amplified in all the samples (FIG. 6). Densitometric scanning (FIG. 7) revealed increase in PPARγ transcripts with percentage of elevation found to be 39.27, 50.50 and 63.63 for 250, 125 and 62.5 μg/mt respectively, which are significantly comparable with standards.

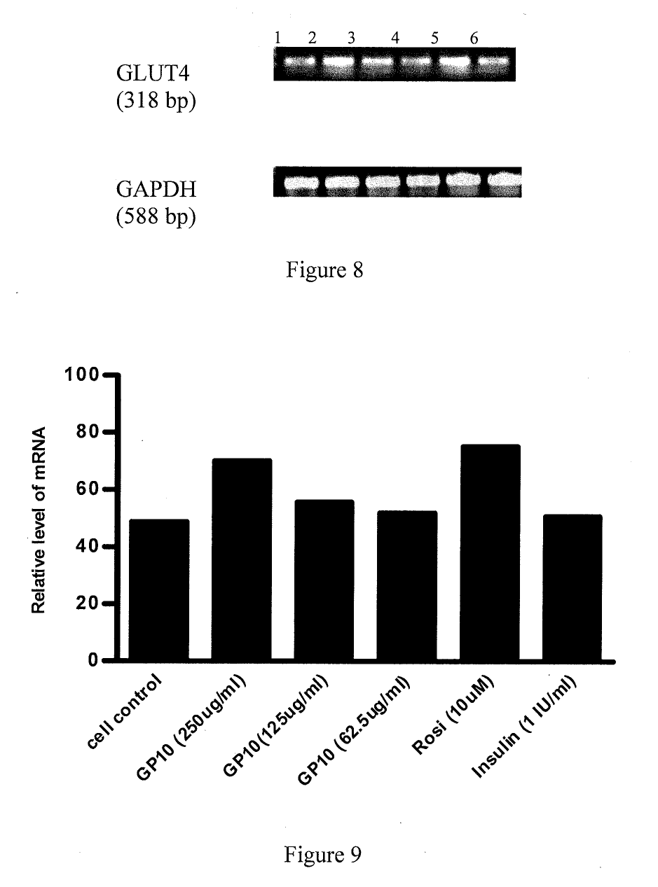

The effect of GP-10 on GLUT4 gene expression was investigated. The RT-PCR analysis showed that treatment of GP-10 up regulated GLUT4 transcript only at higher concentration (FIG. 8). Densitometric analysis showed that, the percentage of gene elevation was found to be 46 at 250 μg/ml, which is comparable with rosiglitazone (FIG. 9). GAPDH was used as internal control (FIG. 8).

### EXAMPLE-9

### Effect of GP-10 on Oral Glucose Tolerance Test in Normal Rats (OGTT)

Male Wistar rats of body weight approximately around 200-250 gms were selected and divided in to 4 groups of eight rats each and the animals were fasted overnight. Group I animals were served as normal control and receive only vehicle (10 ml/kg.p.o), Group II and Group III animals received GP-10 (500 mg/kg and 1000 mg/kg, p.o respectively). Group IV animals were treated with insulin sensitizer rosiglitazone orally (5 mg/kg of body weight). On day 1, overnight fasted animals, one hr after the respective treatment were orally challenged with 2 gm/kg body weight of aqueous glucose solution, The serum glucose levels were checked on 0, 30, 60 and 120 mins after oral glucose challenge. The treatment was continued for 7 days, on day 7, the OGTT was carried as on day 1.

### EXAMPLE-10

### Effect of GP-10 on Fructose Induced Insulin-Resistance and Metabolic Alteration in Rats

Purpose and Rationale: Rats fed with a high-fructose diet (>60% of total calories) provide a useful animal model of insulin resistance and hyperinsulinemia. The sites of fructose-induced insulin resistance are documented to be the liver, skeletal muscle and adipose tissue. These rats also develop a cluster of abnormalities, which include hypertension, hypertriglyceridaemia, oxidative stress and glucose intolerance in addition to hyperinsulinaemia and insulin resistance. This model depicts various metabolic alterations, which are usually observed clinically in people suffering from “metabolic syndrome”

Fructose Enriched diet: A special diet was prepared in such that the fructose content provided 60% of total calories in the diet. The diet was prepared in laboratory with the following composition (g/kg) Casein, high protein—207.0; DL-methionine—3.0; fructose—600.0; lard —50.0; cellulose—79.81; mineral mix—50.0; zinc carbonate—0.04; vitamin mix—10.

Selection of dose of drugs: The drugs were administered once by oral gavage. The dose of GP-10 fixed randomly; one group of animal was provided with GP-10 fortified fructose diet (which received GP-10 approximately 1000 mg/kg body weight/day of animal). For the other groups the dose of GP-10 was fixed as 250 mg/kg, and 500 mg/kg respectively. The dose of standard drug Rosiglitazone was fixed based on the available information in the literature. Rosiglitazone (a standard insulin sensitizer) was supplemented with fructose feed at a dose of 5 mg/kg body weight/day of animal.

Procedure: The male Wistar rats of 200 to 220 gm in body weight were be divided in to six groups of eight each. Group I animals were kept on standard laboratory feed (Tetragon Chemie, Vetcare). Group II to Group VI animals were replaced with high fructose diet. Group II animals served as positive control and receive no treatment, group III and group IV animals were treated with fixed dose of drug GP-10, 250 mg/kg p.o and 500 mg/kg p.o respectively, Group V animals were supplemented with GP-10 fortified fructose diet (1000 mg of GP-10/kg body weight/day of animal). Group VI animals were supplemented with Rosiglitazone in fructose feed at a dose of 5 mg/kg body weight/day of animal.

Animals were maintained on these regimens for 6 weeks; body weight measurements were carried out weekly. At the end of the 6-week of treatment, the rats were fasted overnight and blood of around 3-4 ml was collected under tight ether anesthesia from retro orbital plexus. Blood samples were immediately centrifuged (3000 g for 20 min) and plasma separated for analysis of fasting blood glucose, cholesterol, Triglycerides, LDL, VLDL, HDL and Insulin.

The degree of insulin resistance was estimated by using Homeostasis Model Assessment (HOMA) as an index of insulin resistance:
Insulin (in μU) X glucose (in m mol/L)]/22.5
Note:
1. At the end of 5-week period, two days before final blood collection, the animals were fasted overnight and one hour after the assigned treatments; all the groups of animals were subjected for oral glucose tolerance test as per the standard procedure.
2. The Urine collection for all animals were carried out in the last week of treatment and subjected for measurement of urine volume, sodium, creatinine and uric acid.

### EXAMPLE-11

### Hypoglycemic Activity of GP-10 in Rat Model of Diabetes Induced by Differential Dose of Streptozotocin (a Model of Human Type II Diabetes and (Imperfectly) Treated Type I Diabetes

Around 20 male Wistar rats aged b/w 5 to 6 weeks old were housed in a standard laboratory conditions. They were divided in to 2 groups of 10 each. Group I animals received 0.5M-citrate buffer (pH 4.5) and Group-2 animals were administered with freshly prepared streptozotocin (45 mg/kg) intraperitoneally, which was further followed one week later by an i.p. injection of 35 mg/kg of streptozotocin (solution was prepared in 0.5M-citrate buffer (pH 4.5). This schedule was chosen in order to induce moderate chronic hyperglycemia and thereby to simulate the clinical condition in human Type II diabetes and (imperfectly) treated type I diabetes. The STZ caused persistent moderate hyperglycemia, once the steady state is reached (one week after last dose of STY). The animals were subjected for initial screening of hyperglycemia and the animals which showed basal fasting blood glucose level of around 200 mg/dl were, selected and assigned as treatment group. Group I animals received 10 ml/kg of vehicle (0.5% w/v of CMC) and served as control. Group II animals were treated with GP-10 (500 mg/kg p.o).

0, 60, 120 and 240 minutes post drug administration, small quantity of blood was collected from retro-orbital sinus, serum separated and subjected for estimation of glucose. The treatment was continued for 8 days, on day 8, one hour after the treatment, the blood was collected at different time intervals and subjected for estimation of glucose, as on day-1.

### Statistical Analysis

The values will be expressed as mean±SEM. The results were analyzed statistically using ANOVA to find out the level of significance. The minimum level of significance will be fixed at p<0.05.

### Results

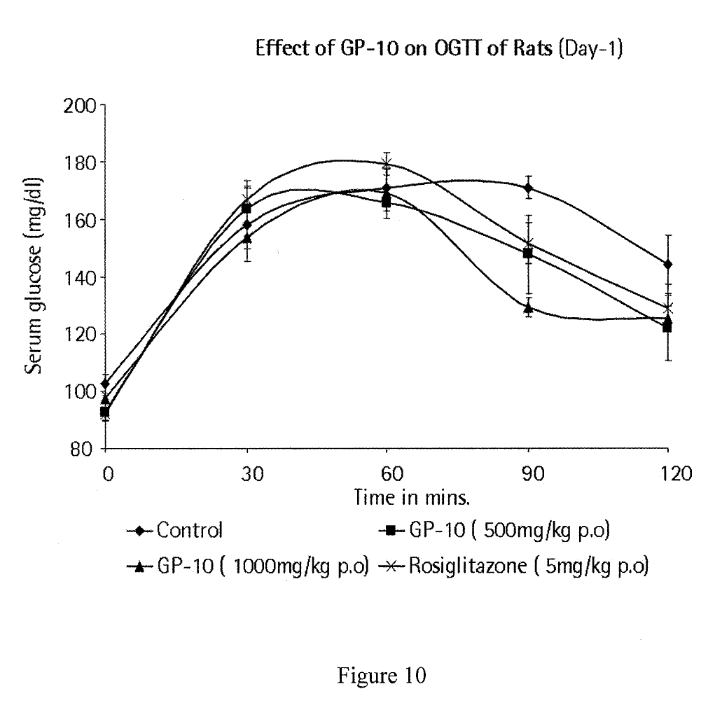

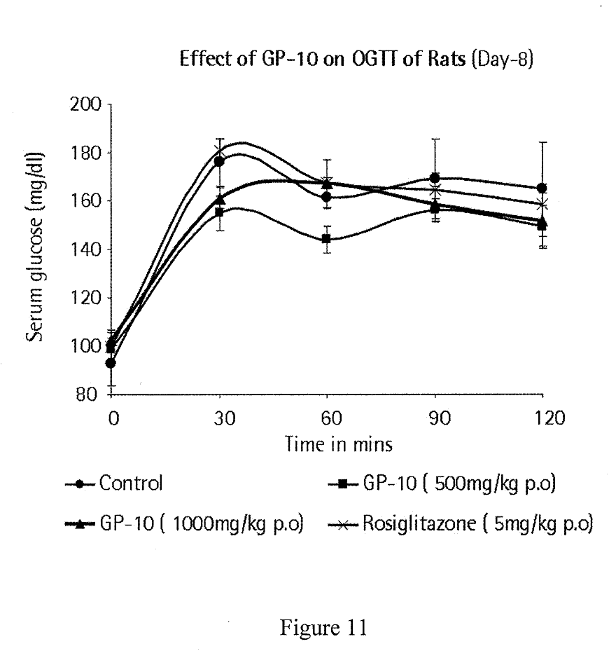

The experimental drug GP-10 was tested for hypoglycemic activity after an oral glucose challenge in normal rats (OGTT). It was found that GP-10 at a dose of 500 mg/kg and 1000 mg/kg body wt p.o doesn't exhibit a statistically significant hypoglycemic activity in both single and multiple doses (8 days), similar results were obtained for a standard drug rosiglitazone, which is a PPAR-gama agonist (FIG. 10 and FIG. 11).

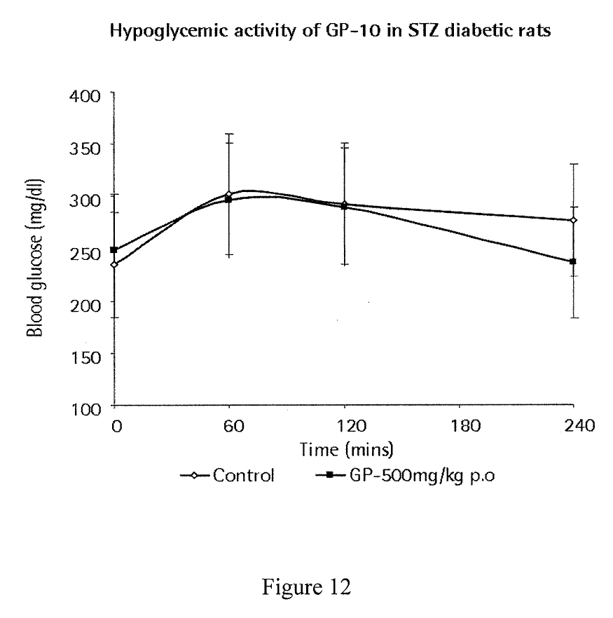

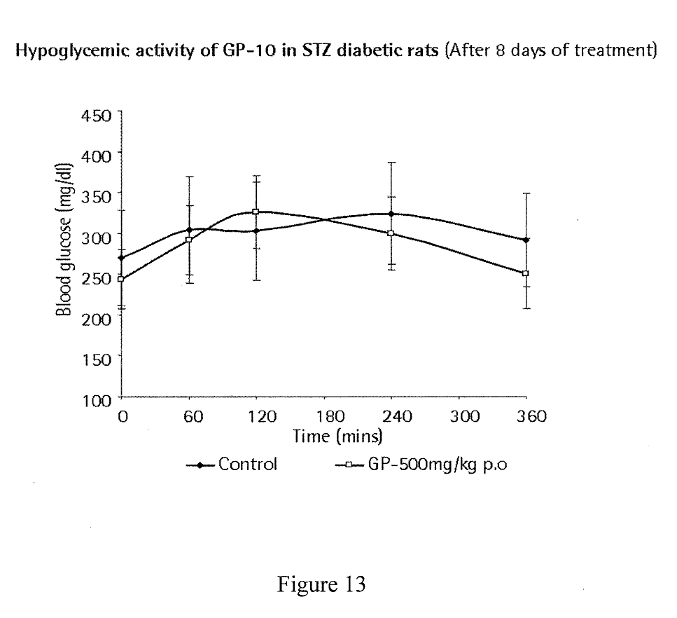

The drug GP-10 was also tested for hypoglycemic activity in STZ diabetic model of rats, where a mild diabetes was induced by administering differential doses of streptozotocin (STZ), the schedule was chosen to induce moderate chronic hyperglycemia, which could simulate the clinical condition in human type II diabetes and (imperfectly) treated type I diabetes. It was found that the drug GP-10 doesn't exhibit significant hypoglycemic activity at 0, 60, 120 and 240 minutes post administration at a dose of 500 mg/kg p.o. Similarly, even multiple doses of GP-10 (for 8 days) fail to show hypoglycemic activity in STZ diabetic rats (FIG. 12 and FIG. 13).

A study was designed in order to investigate, whether the administration GP-10 may improve insulin sensitivity in fructose-fed rats, a well-known model of insulin-resistance, hyperinsulinemia and hypertriglyceridemia. Rosiglitazone (a standard PPAR-gamma agonist and an insulin sensitizer) was included in the study as reference standard (dose of which is approximately, corresponds to a 5 mg/kg, body weight/day). GP-10 was tested at randomly selected doses of 250 mg and 500 mg/kg p.o per day. For one group of animals, the drug GP-10 was administered by supplementing with fructose enriched diet similar to standard drug (1000 mg/kg body weight/day of animal).

During the entire study period, there was no statistically significant change in body weight was observed in any of the groups. At the end of 3^{rd} and 6^{th} week of study, the serum samples obtained were subjected for analysis of various biochemical parameters like total cholesterol, triglycerides, insulin, glucose etc. Serum insulin levels were significantly elevated in animals fed with fructose-enriched diet, indicating hyperinsulinemic state. The insulin resistance index (HOMA) was also found to be significantly increased. Triglyceride (TG) levels were also significantly elevated in fructose fed rats compared to animals, which received normal feed.

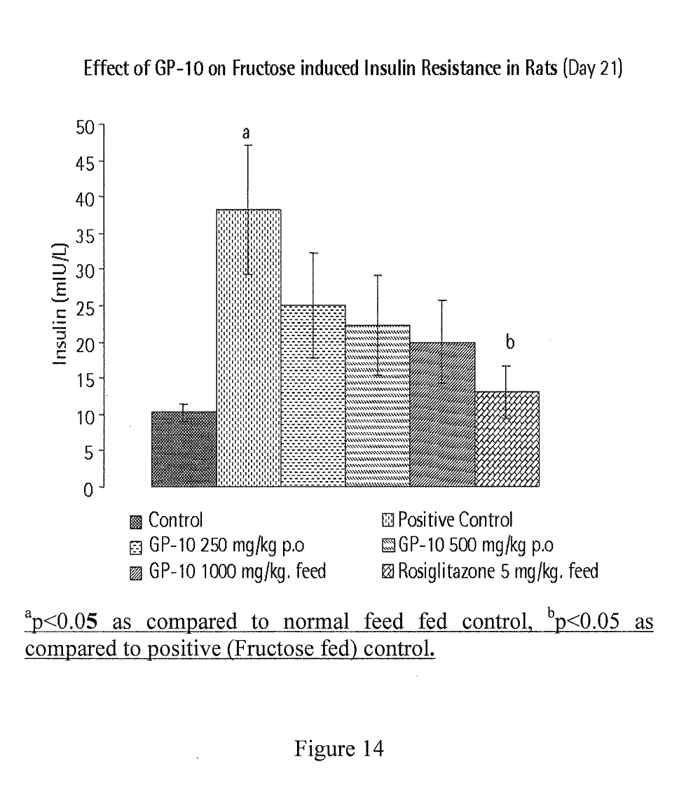

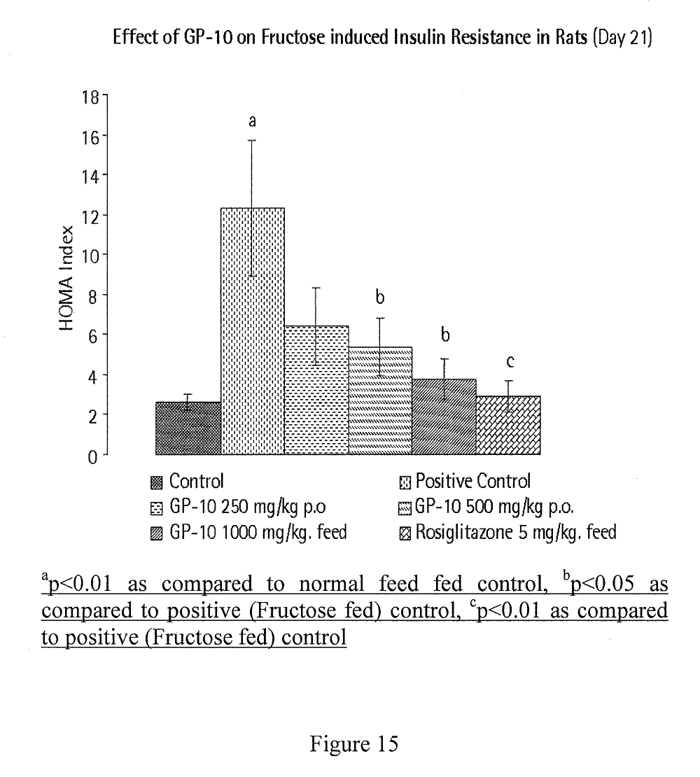

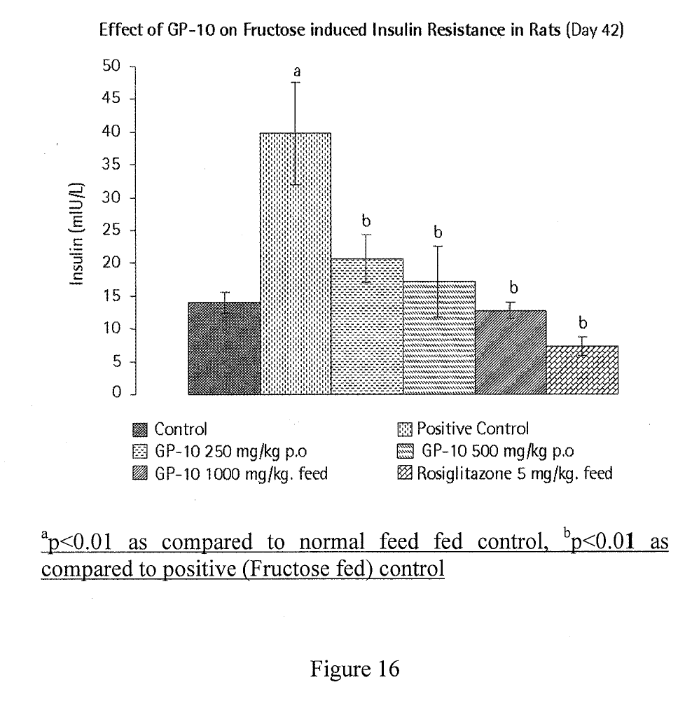

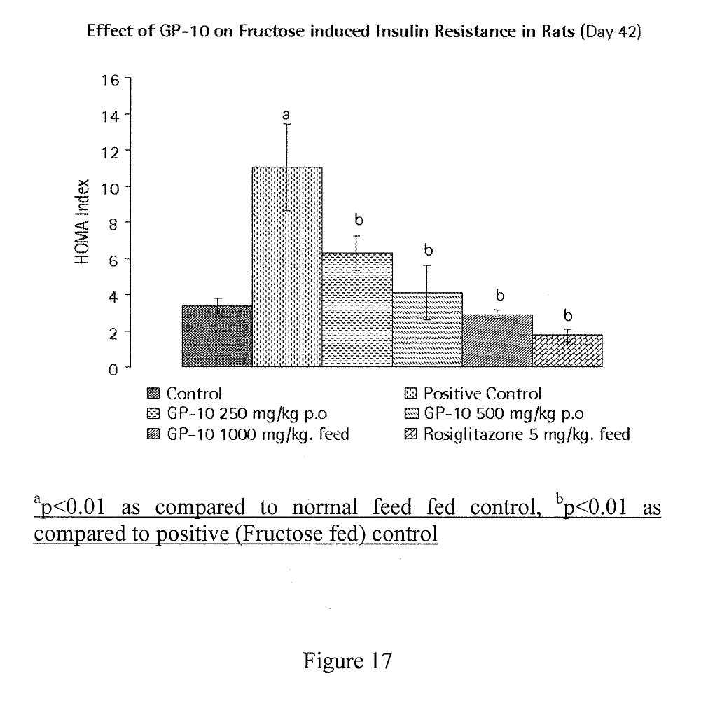

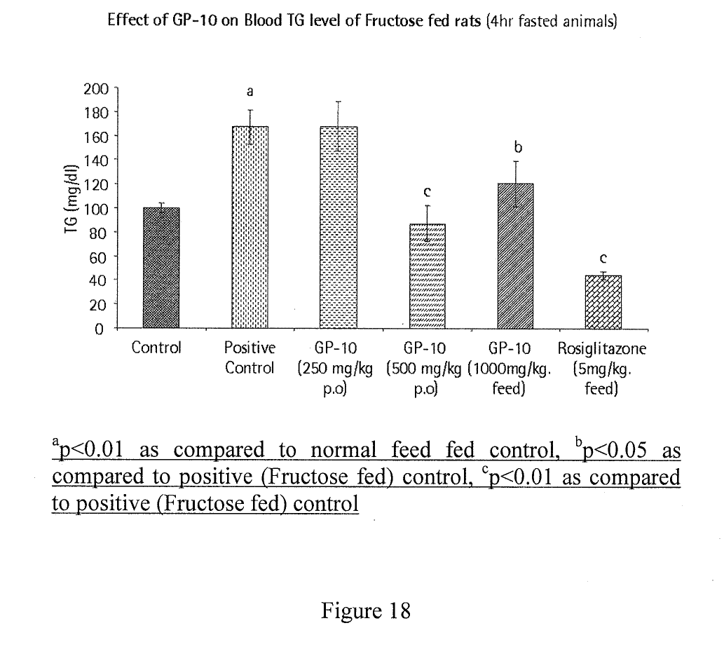

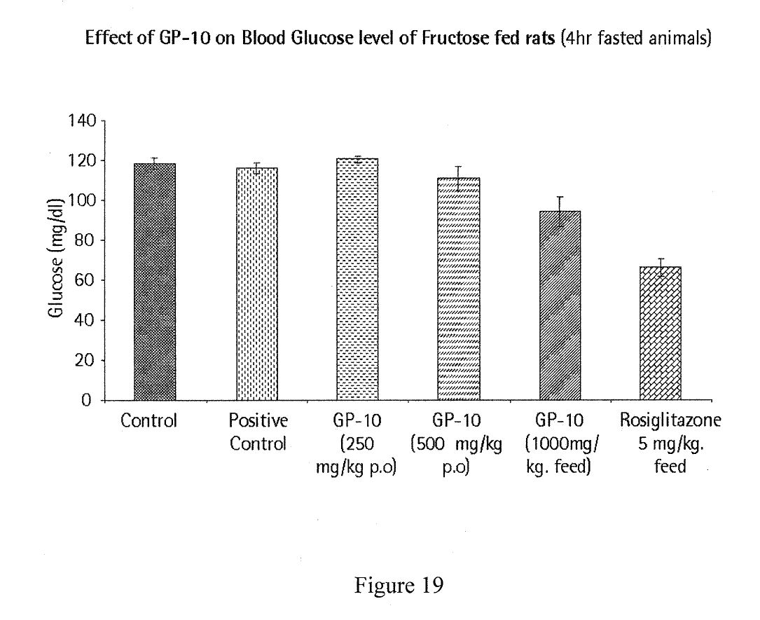

The above elevation of insulin, insulin resistance index, TG were reversed in animals, which received drug GP-10 and rosiglitazone on day 21, but more notable on day 42 (FIG. 14 to FIG. 17). Also, the treatment resulted in significant decrease in serum TG levels but to a lesser extent of serum glucose levels (FIG. 18 to FIG. 19). The GP-10 exhibited the above effect in a dose dependent manner and its activity was comparable to rosiglitazone

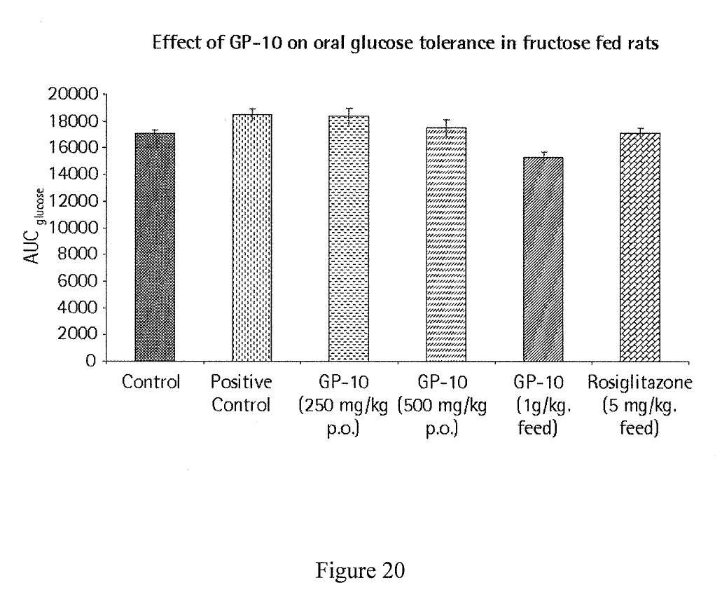

Oral glucose tolerance test (OGTT) was conducted for animals of all the groups during the last week of the study. The results obtained (FIG. 20) showed, the animals that received fructose-enriched diet were glucose intolerant when compared to normal control. We found that supplementation of drugs rosiglitazone and GP-10 decreased the area under curve (AUCC_{glucose}) values indicating the better glucose disposal rate (FIG. 20).

### EXAMPLE-12

### Manufacturing of GP-10 Granules for Tablets (Formula-1, Table-4)

TABLE 4

| S. No | MATERIAL NAME | MG/TAB | | PERCENTAGE |
|---|---|---|---|---|
| 1. | GP-10 (Active) Extract | 500.0 | | 50.0 |
| 2. | Lactose | 250.0 | | 25.0 |
| 3. | Microcrystalline cellulose | 237.5 | | 23.75 |
| 4. | Pre gelatinized starch | 10.5 | | 1.05 |
| 5. | Magnesium stearate | 2.5 | | 0.5 |
| | Total | 1000 | mg | 100 |

### EXAMPLE-13

### Manufacturing of GP-10 Granules for Tablets (Formula-2, Table-5)

TABLE 5

| S. No | MATERIAL NAME | MG/TAB | | PERCENTAGE |
|---|---|---|---|---|
| 1. | GP-10 (Active) Extract | 250.0 | | 41.6 |
| 2. | Lactose | 120.0 | | 20.0 |
| 3. | Microcrystalline cellulose | 215.0 | | 35.9 |
| 4. | Pre gelatinized starch | 12.0 | | 2.0 |
| 5. | Magnesium stearate | 3.0 | | 0.5 |
| | Total | 600 | mg | 100 |

### EXAMPLE-14

### Procedure of Manufacturing GP-10 Granules for Tablets

Step 1: Sift the Excipient through 440. Blend Lactose, Microcrystalline cellulose and Pregelatinized starch.
Step 2: Heat water to 50° C., add GP-Extract, Mix uniformly
Step 3: Granulate Step I with Step II, if required additional quantity of DM water to make wet mass.
Step 4: Sift the wet mass through #8 mesh. Dry in FBD at 70° C.-80° C. to moisture content of 3-4%.
Step 5: Dried granules passed through #16 mesh. Mill oversize granules through 1.5 mm mesh in multi mill. Recheck the moisture.
Step 6: Blend with Magnesium stearate in OGB3 for 5 minutes.
Step 7:The lubricated blend ready for compression into required dimension of tablets

### EXAMPLE-15

### Manufacturing of GP-10 Granules for Capsule (Formula-1, Table-6)

TABLE 6

| S. No | MATERIAL NAME | MG/CAPSULE | | PERCENTAGE |
|---|---|---|---|---|
| 1. | GP-10 (Active) Extract | 100.0 | | 25.0 |
| 2. | Microcrystalline cellulose | 159.2 | | 39.8 |
| 3. | Lactose | 140.0 | | 35.0 |
| 4. | Magnesium stearate | 0.8 | | 0.2 |
| 5. | Water | Q.S | | |
| | Total | 400 | mg | 100 |

### EXAMPLE-16

### Manufacturing of GP-10 Granules for Capsule (Formula-2, Table-7)

TABLE 7

| | | MG PER | | |
|---|---|---|---|---|
| S. No | MATERIAL NAME | DOSAGE | | PERCENTAGE |
| 1. | GP-10 (Active) Extract | 250.0 | | 62.5 |
| 2. | Microcrystalline cellulose | 100.0 | | 25.0 |
| 3. | Lactose | 49.2 | | 12.3 |
| 4. | Magnesium stearate | 0.8 | | 0.2 |
| 5. | Water | Q.S | | |
| | Total | 400 | mg | 100 |

### EXAMPLE-17

### Procedure of Manufacturing GP-10 Granules for Capsules

Step 1: Sift the excipients through #40. Blend Lactose, Microcrystalline cellulose.
Step 2: Heat water to 50° C., add GP-Extract, Mix uniformly
Step 3: Granulate Step I with Step II, if required add additional quantity of DM water to make wet mass.
Step 4: Sift the wet mass through #8 mesh. Dry in FBD at 70° C.-80° C. to moisture content of 3-4%,
Step 5: Dried granules passed through #18 mesh. Mill oversize granules through 2.0 mm mesh in multi mill. Recheck the moisture.

Step 6: Blend with Magnesium stearate in OGB for 5 minutes.
Step 7: The lubricated blend ready for capsule filling in Gelatin/HPMC capsule.

### EXAMPLE-18

### Manufacturing Formula of GP-10 Cream Fable-8

TABLE 8

| S. No. | MATERIAL NAME | PERCENTAGE |
|---|---|---|
| 1. | Carbopol 940 | 0.2 |
| 2. | TEA | 1.0 |
| 3. | Glycerin | 10.0 |
| 4. | Methylparaben | 0.25 |
| 5. | Propyl paraben | 0.05 |
| 6. | Stearic acid | 4.0 |
| 7. | Cetyl alcohol | 0.5 |
| 8. | GP-10 (active) Extract | 1.0 |
| 9. | DM Water | QS |
| | Total | 100 |

### EXAMPLE-19

### Procedure of Manufacturing GP-10 Cream for Topical Application

Step 1: Take half the quantity of water & Glycerin in vessel, disperse carbopol slowly. Heat to 70° C. and add TEA under mixing.
Step 2: Dissolve actives in remaining quantity of water by heating and add step 1
Step 3: Heat 4-7 to 70° C., add to step 1 under mixing. Allow to cool to 40° C. by mixing.

### EXAMPLE-20

### Manufacturing Formula of GP-10 as Gel (Table-9)

TABLE 9

| S. No. | MATERIAL NAME | PERCENTAGE |
|---|---|---|
| 1. | Carbopol 940 | 0.5 |
| 2. | TEA | 0.55 |
| 3. | Glycerin | 10.0 |
| 4. | Methyl Paraben Plain | 0.25 |
| 5. | Propyl Paraben Plain | 0.05 |
| 6. | G.P-10 (active) Extract | 1.0 |
| 7. | DM Water | QS |
| | Total | 100 |

### EXAMPLE-21

### Procedure of Manufacturing GP-10 Gel

Step 1: Take half the quantity of water & Glycerin in vessel, disperse carbopol. Heat to 70° C. and add TEA under mixing.
Step 2: Dissolve completely Methyl and Propyl Paraben in remaining water, which is heated to 80° C. and later dissolve active, add to step 1 under mixing.
Step 3: Allow to cool to 40° C. by mixing.

### EXAMPLE-22

### Manufacturing Formula of GP-10 Syrup (Table-10)

TABLE 10

| S. No. | MATERIAL NAME | PERCENTAGE |
|---|---|---|
| 1. | GP-10 (Active) Extract | 5.0 |
| 2. | Methyl paraben plain | 0.25 |
| 3. | Propyl paraben plain | 0.02 |
| 4. | Sorbitol | 30.0 |
| 5. | Sodium citrate | 0.1 |
| 6. | Citric acid | 0.15 |
| 7. | Disodium EDTA | 0.05 |
| 8. | Water | QS |
| 9. | Xanthan Gum | 0.3 |
| 10. | Propylene Glycol | 4.0 |
| 11. | Mint Flavour | 0.2 |
| 12. | Sodium Saccharin | 0.3 |

| | |
|---|---|
| Total | 100 |

### EXAMPLE-23

### Procedure of Manufacturing GP-10 Syrup

Step 1: Heat water to 85° C., add Parabens, dissolve completely followed by Active, cool to 40° C.
Step 2: Dissolve separately sodium citrate, citric acid, Disodium EDTA and Sodium Saccharine in water separately and add to Step 1.
Step 3: Disperse Xanthan Gum in Propylene glycol and add to Step 1, mix.
Step 4: Add Sorbitol and flavours, make up the volume.

### EXAMPLE-24

### HPLC Standardization of Active Fraction (GP-10/1) and GP-10 Extract

Sample preparation: Both sample GP-10 and GP-10/1 were prepared 1 mg/ml concentration in water and sonicated for 10 minutes and filtered through 0.2 μm syringe filter before injecting the sample.

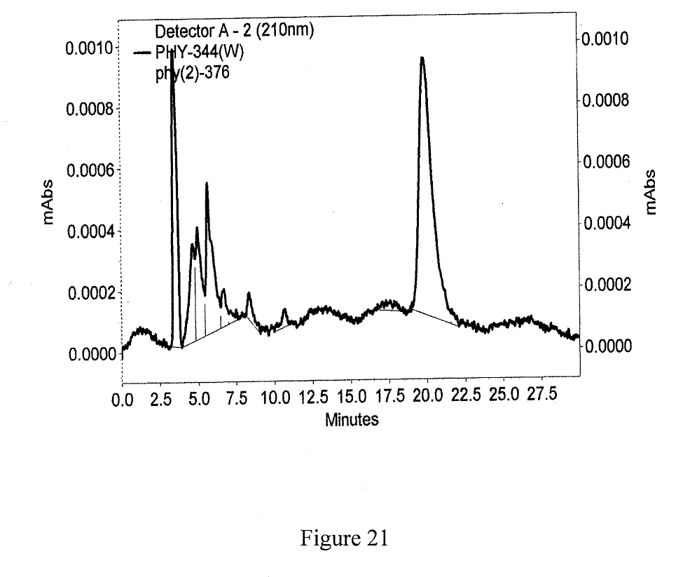

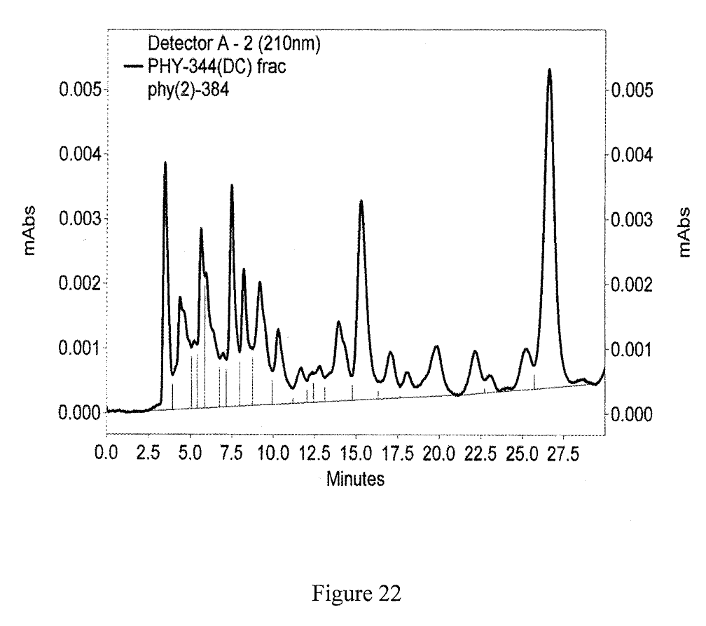

HPLC conditions: Accurately 20 μL of the sample was injected over reverse phase column C18 and run with mobile phase of Buffer (0.1% ortlophosphoric acid in water) at flow rate of 0.8 ml/min. and detected at 210 nm. The chromatogram of the same is given as FIG. 21 (GP-10) and FIG. 22 (GP-10/1).

### Discussion & Conclusion

The water extract (GP-10) of roots of Cipadessa baccifera was found to have potent glucose transport activity in muscle cells. The GP-10 induced glucose transport in muscle cells is comparable with that of rosiglitazone and insulin, which encouraged us to further work on GP-10 as molecular basis of insulin resistance clearly depends on impaired insulin signal transudation with key defects in the glucose transport. PPARγ agonists, a new class of insulin sensitizing drugs has allowed the treatment of insulin resistance associated with Type II diabetes.

Significant glucose uptake activity of GP-10 in muscle cells has given us impetus to study its effect on PPARγ by RT-PCR. Previous studies have also shown that the herbal preparations could effect PPARγ gene expression. Our studies on PPARγ showed that GP-10 has up regulated PPAR gamma gene expression in L6 cells and this finding further suggests that GP-10 has PPAR gamma agonistic activity. GP10 is found to activate PPAR gamma at lower concentration as compared to higher concentration and PPAR gamma activators have shown activities at lower concentration. We further looked into role of GP-10 on GLUT-4 transcription, which is known to facilitate glucose transport in muscle cell. Our findings suggest that GP-10 has elevated GLUT 4 mRNA in L6 muscle cells. Elevated levels of PPARγ mRNA along with enhanced GLUT4 transcription suggested the possible role of PPARγ in the induction of glucose uptake by GP-10. Activation of PPARγ through PPARγ agonists are known to increase the glucose uptake through induction of GLUT4 mRNA.

Therefore, taken together, these findings from in vitro studies show that GP-10 has potent glucose transport capacity and modulates the expression of PPAR gamma and GLUT 4 in L6 cells. Among the different fractions yielded through purification of GP-10, GP-10/1 and ESG-81 exhibited potent glucose uptake properties in L6 muscle cells.

Analysis of animal experimental results of various experiments, have shown that water extract of roots of Cipadessa baccifera (GP-10) doesn't have hypoglycemic activity in both normal and STZ diabetic rats similar to that of Rosiglitazone, which is a PPAR-gamma agonist and a well-known insulin sensitizer. The drug GP-10 reversed the hyperinsulinemia insulin resistance, hypertriglyceridemia and glucose intolerance in high fructose fed rats, which is a non-genetic experimental model of metabolic syndrome. The observed effect of GP-10 was found to be dose dependent and comparable to that of Rosiglitazone.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

We claim:

1. A novel herbal composition for treating Type-II diabetes comprising extract of roots of Cipadessa baccifera and pharmaceutically acceptable excipients.

2. The herbal composition according to claim 1, wherein said extract of roots of Cipadessa baccifera is obtained by employing a solvent or solvent system selected from a group comprising organic solvents and/or water.

3. The herbal composition according to claim 2, wherein the organic solvent is preferably selected from n-hexane, chloroform, dichloromethane, ethyl acetate, acetone or methanol, ethanol or butanol alone or in combination thereof.

4. The herbal composition according to claim 2, the solvent is preferably butanol or water alone or in combination.

5. The herbal composition according to claim 1, wherein the pharmaceutical excipients is selected from the group comprising filler, binder, disintegrating agent, lubricant, preservative, sequestering agent, thickening agent, solvent, flavoring agent, sweetening agent, humectant, rheology modifier, emollient, emulsifying agent and/or water.

6. The herbal composition according to claim 1, wherein the composition comprising effective amount of water extract of roots of Cipadessa baccifera is about 100 mg to about 1000 mg.

7. The herbal composition according to claim 1, wherein said composition is prepared by a method comprising:
(i) extracting the roots of Cipadessa baccifera employing a suitable solvent or solvent system;
(ii) filtering the resultant plant extract from above step by employing suitable filtering aid;
(iii) the resultant filtrate of the above step (ii) is spray dried or freeze dried to obtain free flowing dry extract powder;
(iv) producing a anti-diabetic herbal composition by mixing therapeutically effective amount of said dry extract powder resulted from the step (iii) and pharmaceutically acceptable excipients.

8. The process according to claim 7, wherein said extraction is preferably performed by employing percolation or hot-soxhlation method.

9. The process according to claim 7, wherein said solvent or solvent system selected from any organic solvent and water, preferably selected from n-hexane, chloroform, dichloromethane, ethyl acetate, acetone, methanol or ethanol alone or in combination thereof, more preferably water.

10. The herbal composition according to claim 1, wherein said composition is formulated in any suitable delivery form, preferably in the form of tablet, capsules, syrup, cream and gel.

11. The herbal composition according to claim 1, wherein said extract of roots of Cipadessa baccifera is characterized by having biologically active fraction capable of reducing the glucose, triglyceride levels in blood or other complications related to the Type-II diabetes and its secondary complications thereof.
